(19) United States
(12) Patent Application Publication
(10) Pub. No.: US 2026/0000542 A1
(43) Pub. Date: Jan. 1, 2026

(54) X-RAY AND METAL DETECTABLE BANDAGE

(71) Applicant: Aero Healthcare (US), LLC, Valley Cottage, NY (US)

(72) Inventors: Brielle Ovenden, Armidale NSW (AU); Murray Scott, Armidale NSW (AU)

(21) Appl. No.: 19/248,687

(22) Filed: Jun. 25, 2025

Related U.S. Application Data
(60) Provisional application No. 63/664,434, filed on Jun. 26, 2024.

(51) Int. Cl. A61F 13/00

(52) U.S. Cl. CPC A61F 13/00051

(57) ABSTRACT

A detectable bandage and methods of manufacturing thereof are disclosed. In some embodiments, an x-ray detectable layer comprising an x-ray detectable material and a matrix and a metal detectable layer comprising a metal detectable material are disposed between a backing layer and a pad of a bandage. The x-ray detectable material may be combined with the matrix to form a thin film that is flexible to conform to a surface to which the bandage is applied. The x-ray detectable layer may be approximately a same size and a shape as the pad providing a large surface area and thickness that is highly flexible and comprises a density of x-ray detectable material that is detectable by standard x-ray detecting methods.

### RELATED ART

This non-provisional patent application claims priority benefit, with regard to all common subject matter, of earlier-filed U.S. Provisional Patent Application No. 63/664,434 filed on June 26, 2024, and entitled "X-RAY AND METAL DETECTABLE BANDAGE." The identified earlier-filed provisional patent application is hereby incorporated by reference in its entirety into the present application.

### BACKGROUND

### 1. Field

Embodiments of the present disclosure generally relate to x-ray and metal detectable bandages. More specifically, embodiments of the present disclosure relate to materials and configurations of x-ray and metal detectable bandages.

### 2. Related Art

Typical detectable bandages include specific materials added to bandages that may be detectable by x-ray and/or metal detecting methods. These detectable bandages are typically used in food processing and food packing industries. People working with, or in close proximity to, exposed food utilize detectable bandages to cover wounds. If these bandages become detached from the wearer, the bandages may still be found by x-ray and metal detecting techniques further down the line during the food processing and packaging. Detecting these detectable bandages is an extremely important step in the food preparation and packaging process as it prevents or reduces the likelihood of food contamination.

Detectable bandages have certain drawbacks. Adding x-ray and/or metal detectable material to bandages commonly compromises the integrity of the bandage causing the bandage to fall off of the wearer more easily. Typically, x-ray and/or metal detectable material is added in a manner that results in lower elasticity and flexibility of the bandage. Furthermore, manufacturing a detectable bandage is typically more expensive than standard non-detectable bandages. What is needed is a detectable bandage that is more reliable, adheres longer, and has a higher detectability that can be manufactured at or below the cost of current typical bandages.

### SUMMARY

Embodiments of the present disclosure solve the above-mentioned problems by providing an x-ray and metal detectable bandage with higher elasticity and flexibility than current detectable bandages at lower cost. The combination of flexibility and elasticity, as described below, provides a detectable bandage that decreases the likelihood of detaching from the user over current bandages. Furthermore, the addition of the detectable material, described in embodiments herein, increases the likelihood of detection if the detectable bandage becomes detached from the user.

In some aspects, the techniques described herein relate to a detectable bandage, the detectable bandage including an elastic backing layer, a pad configured to be applied to a wound of a user contacting the user on a front side and including a back side opposite the front side, a metal detectable layer including a metal detectable material disposed between the elastic backing layer and the pad; an x-ray detectable layer including an x-ray detectable material and a matrix, the x-ray detectable layer disposed between the elastic backing layer and the pad adjacent the metal detectable layer, wherein the x-ray detectable layer covers approximately all of an area of the back side of the pad, and an adhesive on the elastic backing layer configured to contact and adhere to a skin of the user to hold the detectable bandage on the skin.

In some aspects, the techniques described herein relate to a detectable bandage, wherein the metal detectable layer is disposed adjacent the elastic backing layer and the x-ray detectable material is disposed between the metal detectable layer and the pad.

In some aspects, the techniques described herein relate to a detectable bandage, wherein the x-ray detectable material is barium sulfate.

In some aspects, the techniques described herein relate to a detectable bandage, wherein the matrix includes ethylene-vinyl acetate.

In some aspects, the techniques described herein relate to a detectable bandage, wherein a thickness of the x-ray detectable layer is approximately 0.33 millimeters.

In some aspects, the techniques described herein relate to a detectable bandage, wherein the metal detectable material is a first metal detectable material, and further including a second metal detectable material disposed in the elastic backing layer.

In some aspects, the techniques described herein relate to a detectable bandage, wherein the metal detectable material includes tin.

In some aspects, the techniques described herein relate to a detectable bandage, wherein the x-ray detectable material covers at least a portion of the elastic backing layer outside of the pad.

In some aspects, the techniques described herein relate to a detectable bandage, including an elastic backing layer, a pad configured to be applied to a wound of a user, a metal detectable layer including a metal detectable material disposed between the elastic backing layer and the pad, an x-ray detectable layer including an x-ray detectable material and a matrix, the x-ray detectable layer disposed between the elastic backing layer and the pad adjacent the metal detectable layer, wherein the x-ray detectable layer includes barium sulfate, and an adhesive on the elastic backing layer configured to contact and adhere to a skin of the user to hold the detectable bandage on the skin.

In some aspects, the techniques described herein relate to a detectable bandage, wherein the metal detectable layer is disposed adjacent the elastic backing layer and the x-ray detectable material is disposed between the metal detectable layer and the pad.

In some aspects, the techniques described herein relate to a detectable bandage, wherein the matrix includes ethylene-vinyl acetate.

In some aspects, the techniques described herein relate to a detectable bandage, wherein the x-ray detectable material covers approximately an entire surface area of a back side of the pad.

In some aspects, the techniques described herein relate to a detectable bandage, wherein a thickness of the x-ray detectable layer is approximately 0.33 millimeters.

In some aspects, the techniques described herein relate to a detectable bandage, wherein the metal detectable material includes tin.

In some aspects, the techniques described herein relate to a detectable bandage, including an elastic backing layer, a pad configured to be applied to a wound of a user, a metal detectable layer including a metal detectable material disposed between the elastic backing layer and the pad, an x-ray detectable layer including an x-ray detectable material and a matrix including ethylene-vinyl acetate (EVA), the x-ray detectable layer disposed between the elastic backing layer and the pad adjacent the metal detectable layer, wherein the x-ray detectable layer includes barium sulfate, and an adhesive on the elastic backing layer configured to contact and adhere to a skin of the user to hold the detectable bandage on the skin.

In some aspects, the techniques described herein relate to a detectable bandage, wherein the metal detectable layer is disposed adjacent the elastic backing layer and the x-ray detectable material is disposed between the metal detectable layer and the pad.

In some aspects, the techniques described herein relate to a detectable bandage, wherein the x-ray detectable material covers approximately an entire surface area of a back side of the pad.

In some aspects, the techniques described herein relate to a detectable bandage, wherein a thickness of the x-ray detectable layer is in a range of 0.25 to 0.40 millimeters.

In some aspects, the techniques described herein relate to a detectable bandage, wherein the metal detectable material includes tin foil.

In some aspects, the techniques described herein relate to a detectable bandage, wherein the x-ray detectable layer is an isotropic mixture of the EVA and the barium sulfate.

This summary is provided to introduce a selection of concepts in a simplified form that are further described below in the detailed description. This summary is not intended to identify key features or essential features of the claimed subject matter, nor is it intended to be used to limit the scope of the claimed subject matter. Other aspects and advantages of the present disclosure will be apparent from the following detailed description of the embodiments and the accompanying drawing figures.

### BRIEF DESCRIPTION OF THE DRAWINGS

Embodiments of the present disclosure are described in detail below with reference to the attached drawing figures, wherein:

FIG. 1 illustrates an embodiment of a detectable bandage;

FIG. 2 illustrates various layers of the detectable bandage;

FIGS. 3A-3D depict exemplary configurations of the detectable bandage; and

FIG. 4 depicts a flow chart illustrating a process of manufacturing and assembling the layers of the detectable bandage of FIG. 1.

The drawing figures do not limit the present disclosure to the specific embodiments disclosed and described herein. The drawings are not necessarily to scale, emphasis instead being placed upon clearly illustrating the principles of the present disclosure.

### DETAILED DESCRIPTION

The following detailed description of embodiments of the present disclosure references the accompanying drawings that illustrate specific embodiments in which the present disclosure can be practiced. The embodiments are intended to describe aspects of the present disclosure in sufficient detail to enable those skilled in the art to practice the present disclosure. Other embodiments can be utilized, and changes can be made without departing from the scope of the present disclosure. Therefore, the following detailed description is not to be taken in a limiting sense. The scope of embodiments of the present disclosure is defined only by the appended claims, along with the full scope of equivalents to which such claims are entitled.

In this description, references to "one embodiment," "an embodiment," or "embodiments" mean that the feature or features being referred to are included in at least one embodiment of the technology. Separate reference to "one embodiment," "an embodiment," or "embodiments" in this description do not necessarily refer to the same embodiment and are also not mutually exclusive unless so stated and/or except as will be readily apparent to those skilled in the art from the description. For example, a feature, structure, or act described in one embodiment may also be included in other embodiments but is not necessarily included. Thus, the technology can include a variety of combinations and/or integrations of the embodiments described herein.

Generally, embodiments of the current disclosure provide x-ray detectable and metal detectable bandages, generally referenced herein as "detectable bandages" for use in covering wounds on human skin. The detectable bandages may be applied to the skin for covering wounds and, in some embodiments, providing medicine to the wounds. The x-ray detectable material and metal detectable material may generally be referenced as "detectable material." The detectable material may be x-ray detectable, metal detectable, and a combination of x-ray detectable and metal detectable. In some embodiments, the detectable material may be woven into fabric of the bandage, added into the material matrix (described below) such as plastic before forming, added with an adhesive, and/or provided by other methods described herein. The detectable material may be added in quantities such that the detectable material may be detected by any metal and x-ray detecting processes typically used in any industry, but specifically in the food industry (e.g., food packing, food service, transportation, etc.).

Generally, the detectable bandage described herein may be used in any industry where it is necessary to detect a bandage that has become detached from the user. For example, the detectable bandage may be used in the food packing industry. As describe above, a worker with a typical bandage working in contact with food may lose their bandage while working. The bandage may end up falling into the food. As it is a health hazard to have the bandage in the food, the bandage must be found before any food that has become contaminated is packaged and delivered. Typically, at stages of food processing, the food goes through metal and x-ray detection machines to ensure that the food is not contaminated. If a bandage has become detached from the worker and falls into the food, the bandage should be detected at these detection stages. The detectable bandage described herein may also be used in the food service industry (e.g., restaurants, concessions, food trucks, vendors, fast food chains, etc.). The detectable bandage described in detail below, may provide high detectability and adhesion while maintaining low costs.

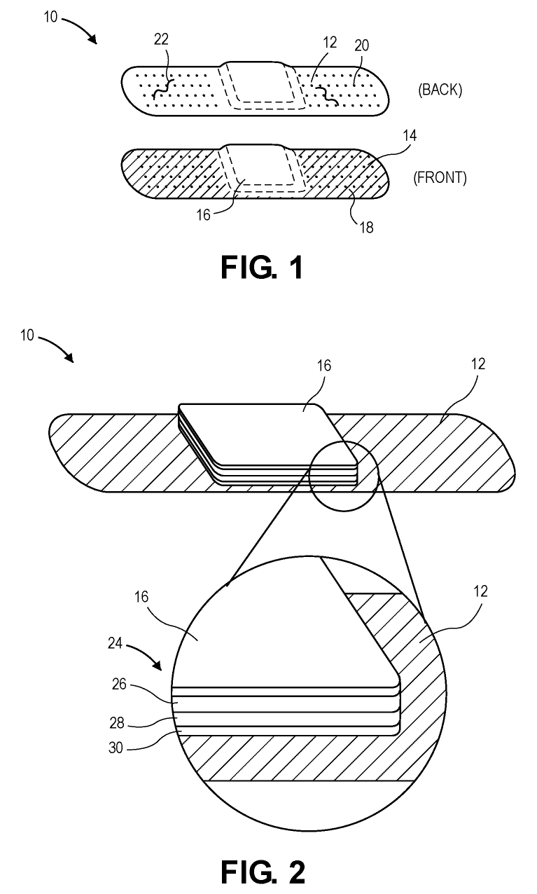

FIG. 1 depicts an exemplary detectable bandage 10 comprising a backing 12, adhesive layer 14, and a pad 16 as shown. Intermediate layers are shown in FIG. 2 and discussed in detail below. In some embodiments, backing 12 comprises elastic, woven and/or unwoven fabric, plastic, cloth, gauze, latex and the like. Backing 12 may provide support for the other layers of detectable bandage 10 as well as elasticity and flexibility to conform to a shape of the surface to which detectable bandage 10 is applied. Backing 12 may or may not be absorbent and may, in some embodiments, be airtight and/or watertight. Alternatively, backing 12 may comprise backing holes 20 such that detectable bandage 10 is breathable and provides less resistance to elasticity and flexibility. Therefore, the detectable bandage 10 may stretch and conform to non-flat surfaces provided by a wearer's skin. This allows detectable bandage 10 to be applied to various non-flat surfaces.

Furthermore, in some embodiments, various layers of the detectable bandage 10 may be flexible to conform to the variable surfaces and movements of the user's skin and skeletal structure. The combination of flexibility and elasticity decreases the likelihood over current detectable bandages of detectable bandage 10 detaching from the user. Furthermore, the addition of the detectable material, described in embodiments herein, increases the likelihood of detection if detectable bandage 10 becomes detached from the user.

In some embodiments, backing 12 may comprise detectable material 22. Detectable material 22 may be an x-ray detectable and/or metal detectable thread that may be woven into the fabric of backing 12. Using metal detectable material as an example, the metal detectable material may be any metal and/or combinations of fabric threads or plastics mixed with metal powder. The detectable threads may provide an amount of metal along with a quantity of threads such that the amount of metal in each bandage is detectable by standard metal detection processes. In some embodiments, metal detectable threads may be provided in combination with metal detectable layers (described below) such that a total quantity of metal in detectable bandage 10 is detectable by standard metal detection processes.

In some embodiments, detectable material 22 may be woven into backing 12 or applied with an adhesive. As described above, detectable material 22 may be a metal or metal-infused thread that is detectable by standard metal detection methods. In some embodiments, detectable material 22 may be laminated on backing 12 and/or detectable metal may be added into backing 12 when backing 12 is processed (e.g., woven, mixed, etc.). In some embodiments, backing 12 may comprise polyurethane. The polyurethane may comprise blue die for visibility as well as metallic powder. As such, backing 12 may be plastic and detectable my standard metal detecting methods. In this case, detectable material 22 may be mixed into the backing material matrix before forming by extrusion, pressing, rolling, and/or other means of forming. In some embodiments, detectable material 22 may be added after forming by pressing detectable material 22 into backing material or attaching detectable material 22 by an adhesive or laminate. Detectable material 22 may be used in combination with the below described embodiments including x-ray detectable layer 26 and metal detectable layer 28.

Detectable bandage 10 may comprise backing adhesive layer 14. In some embodiments, backing adhesive layer 14 may comprise a backing adhesive 18 applied directly or indirectly to backing 12. In some embodiments, backing adhesive 18 comprises methacrylate, epoxy diacrylates, and the like. Backing adhesive 18 may be used to attach detectable bandage 10 to a skin of the user. Detectable bandage 10, as shown includes backing adhesive layer 14 on the "front," as the front is described herein as the side of detectable bandage 10 in contact with the skin of the user. The "back" is opposite the front as shown.

Detectable bandage 10 includes pad 16. In some embodiments, pad 16 comprises woven or unwoven fabric providing absorption characteristics for absorbing fluids from the wound of the user. Pad 16 may be sized to fit a width of backing 12. In some embodiments, a pad width of pad 16 may be approximately a backing width of backing 12 and an adhesive may be applied to any layers exposed to the skin of the user. Alternatively, the width of pad 16 may be slightly less than the backing width such that a portion of backing 12 and backing adhesive layer 14 is exposed on each side of pad 16 to the user's skin. This provides more adhesive in contact with the user's skin directly around pad 16. For example, the pad may be a percentage of surface area of the backing (e.g., 10%, 15%, 20%, 30%, and the like). In some embodiments, pad 16 may be square, rectangular, oval, or any other shape that may cover various wounds. The shape and size of pad 16 may be optimized based on wound types (e.g., cuts, scrapes, puncture, and the like) as well as material costs, and amount of adhesive/pad exposed to the skin of the wearer. Furthermore, pad 16 may be adhered to backing 12 by adhesives through several intermediate layers described below. In some embodiments, pad 16 may comprise cotton, rayon, polyester woven and nonwoven fibers.

In some embodiments, the detectable bandage may be a specifically selected color. The detectable bandage 10 may be any color including colors that are not typically found in food. For example, blue is not a color that is typically found in food. As such, in some embodiments, detectable bandage 10 may be blue to contrast with food such that detectable bandage 10 may be easily seen by workers inspecting the food before packaging and/or service. In some embodiments, detectable bandage 10 may be red, yellow, blue, or any combination to generate any pigment color.

In some embodiments, medicine may be provided by pad 16 to disinfect and aid in healing the wound. Accordingly, any standard medicine (e.g., antiseptic, antimicrobial, antibiotic, anti-itch, hypoallergenic, steroid, and the like) may be provided on pad 16 to be applied to the wound during use. Furthermore, a porous plastic may be added to the surface of pad 16 to resist sticking when detectable bandage 10 is removed from the wound.

FIG. 2 depicts bandage layers 24 of detectable bandage 10. In some embodiments, bandage layers 24 comprise backing 12, adhesive layer 14, pad 16, as well as x-ray detectable layer 26, metal detectable layer 28, and adhesive layer 30, which in embodiments, is also provided between metal detectable layer 28 and x-ray detectable layer 26, and between x-ray detectable layer 26 and pad 16. In some embodiments, x- ray detectable layer 26 and metal detectable layer 28 may be switched such that metal detectable layer 28 is positioned between pad 16 and x-ray detectable layer 26; however, the exemplary configuration shown FIG. 2 in is used as an example for description herein.

In some embodiments, x-ray detectable layer 26 comprises x-ray detectable material. The x-ray detectable material may comprise any material that may be detectable by standard x-ray detecting methods and x-ray inspection systems. In some embodiments, the x-ray detectable material may be plastic, hard plastic, silicone, and the like. In some embodiments, the x-ray detectable material is barium sulfate.

In some embodiments, x-ray detectable layer 26 may comprise x-ray detectable material provided in a matrix. The matrix may include any non-x-ray detectable medium, or x-ray detectable medium, supporting the x-ray detectable material (e.g., barium sulfate). In some embodiments the matrix may be a plastic, epoxy, and the like. The matrix may be any material that may be formed into a size and shape suitable for positioning proximate to the pad 16 as x-ray detectable layer 26. Furthermore, the matrix may include at least a large enough quantity of the x-ray detectable material to be detectable by standard x-ray detecting techniques. In some embodiments, the matrix may be ethylene-vinyl acetate (EVA) and the x-ray detectable material may be barium sulfate.

The matrix (e.g., EVA) of x-ray detectable layer 26, in some embodiments, provides flexibility with a specified density of x-ray detectable particles of the x-ray detectable material to be detected by standard methods. X-ray detectable material (e.g., barium sulfate) may be added to the EVA, for example, before the EVA is formed. In some embodiments, the EVA-barium sulfate mixture may be heated and mixed. The mixture may then be formed (e.g., extruded, pressed, etc.) into an extremely thin and flexible film while retaining a density of x-ray detectable particles to be detectable by standard x-ray detecting processes. The quantities of EVA-barium sulfate mixture provided here provide an optimal flexibility while maintaining a quantity of barium sulfate that is x-ray detectable.

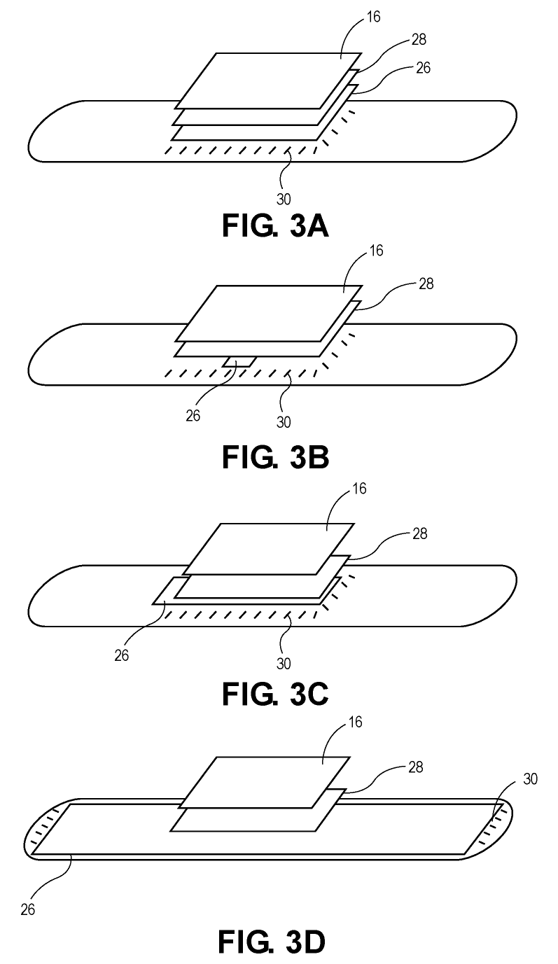

Furthermore, the quantities of x-ray detectable material listed above may be based on the size and shape of x-ray detectable layer 26. Similarly, or alternatively, the size and shape of x-ray detectable layer 26 may be based at least in part on the size and shape of pad 16. As such, the quantities of x-ray detectible material in the matrix may be based on the size of pad 16, which may be provided in various sizes and shapes. The surface area of a side of x-ray detectable layer 26 adjacent pad 16 may be, in some embodiments, approximately equal to the surface area of pad 16 comprising approximately the same size and shape. For example, pad 16 may be square and, likewise, x-ray detectable layer 26 may be square covering the entirety of the adjacent side of pad 16. So, if the surface area of x-ray detectable layer 26 and pad 16 are approximately equal, the amount of barium sulfate in x-ray detectable material is defined by the thickness of x-ray detectable layer 26 and the density/ration of barium sulfate to EVA in the mixture. Therefore, a minimum detectible barium sulfate may be required based on the volume of x-ray detectable layer 26. As such, an optimal balance between flexibility and detectability defines the thickness of the x-ray detectable layer 26. It should be noted that various sizes and shapes of pad 16, x-ray detectable layer 26, and other layers may be provided. For example, the various shapes may include circular, oval, polygonal, or any abstract shape. Furthermore, any sizes of the shapes may be provided. Various configurations of the size and shape of bandage layers 24 are shown in FIGS. 3A-3C and discussed in detail below.

In some embodiments, the size and shape of x-ray detectable layer 26 may be based at least in part on the quantity (described above) of the x-ray detectable material in x-ray detectable layer 26. In some cases, a minimum amount of x-ray detectable material may be required to be detected. Furthermore, in some embodiments, a margin of safety may be applied to provide a higher confidence that x-ray detectable material will be detected. For example, the minimum amount of material required for detection by standard methods may be multiplied by a safety margin of 1.1, 1.2, 1.5, 2.0 or the like to provide an amount of x-ray detectable material that per area or volume of x-ray detectable layer 26 has a high likelihood of being detected. As described above, the quantity of x- ray detectable material in x-ray detectable layer 26 may be based on the volume of x-ray detectable layer 26 to reach the minimum detectable quantity. The minimum detectible quantity may then be multiplied by the safety margin.

In some embodiments, the density of x-ray detectable material in x-ray detectable layer 26 may be based at least in part on the flexibility of x-ray detectable layer 26. An amount of x-ray detectable material may be added to provide a quantity of x-ray detectable material to matrix such that a desirable flexibility of x-ray detectable layer 26 is maintained while providing enough x-ray detectable material to be easily detected by standard x-ray detecting techniques. As a volume of x-ray detectable layer 26 may be known based on the size and shape of pad 16 and a thickness of x-ray detectable layer 26, an amount of x-ray detectable material may be added to arrive at the desired density.

In some embodiments, the size and shape of x-ray detectable layer 26 may be based on a combination of the above-described parameters. For example, it may be desirable to provide as much flexibility to detectable bandage 10 as possible. Accordingly, it may be desirable to provide as much flexibility to x-ray detectable layer 26 as possible. In the case of a plastic matrix infused with x-ray detectable material, the thinner the matrix the more flexible x-ray detectable layer 26 may be. Therefore, it may be desirable to make x-ray detectable layer 26 as thin as possible. In some embodiments, x-ray detectable layer 26 may span approximately 100% of the surface area of pad 16 covering the same size and shape as shown in FIG. 2 . Accordingly, the thickness of x-ray detectable layer 26 and the density of the x-ray detectable material to matrix may be optimized to maximize flexibility of x-ray detectable layer 26 while constraining a minimum amount of x-ray detectable material. In some embodiments, the thickness and density may be based on the safety factor increasing detectability described above.

As described above, in some embodiments, x-ray detectable layer 26 may comprise barium sulfate and the matrix EVA. In some embodiments, barium sulfate mixed with EVA provides a thin film of approximately the same width and length of pad 16 and the thickness of approximately 0.33 millimeters (mm) while maintaining a density of barium sulfate such that detectable bandage 10 is detectable by standard x-ray detecting processes. In some embodiments, a thickness range may be between 0.25 and 0.40 mm. Furthermore, the density of barium sulfate in EVA may provide detectability at high speeds during food production and packing while not limiting the flexibility of detectable bandage 10. In some embodiments, the thin flexible film of x-ray detectable layer 26 may not significantly reduce the overall flexibility of detectable bandage 10. As such, detectable bandage 10 remains attached to the user's skin for longer periods and under harsher conditions than other current detectable bandages.

In some embodiments, metal detectable layer 28 may be provided between x- ray detectable layer 26 and backing 12. Metal detectable layer 28 may comprise an inert material with metal detectable material infused as described above, or metal detectable layer 28 may comprise a metal detectable material such as, for example, a thin metal sheet comprising tin, aluminum, copper, and the like.

As described above, the large area covered by x-ray detectable layer 26 and the relatively high density of x-ray detectable material relative to typical bandages, may provide a high likelihood of detectability of detectable bandage 10 while maintaining desired flexibility. Furthermore, the high surface area of x-ray detectable layer 26 provides additional benefits. For example, in some food preparation processes cutting is required. In the event that detectable bandage 10 falls into food and goes through the cutting process, given the large area covered by the x-ray detectable layer 26 and the relatively high density of the x-ray detectable material as described above, the high likelihood of detecting cut pieces of detectable bandage 10 is maintained.

FIGS. 3A-3C depict additional configurations of detectable bandage 10. In FIGS. 3A-3C, x-ray detectable layer 26 and metal detectable layer 28 are switch from the configuration shown in . The configurations shown in FIGS. 3A-3C illustrate that bandage layers 24 may be various shapes and sizes and in various combinations. depicts an exemplary configuration comprising x-ray detectable layer 26 approximately the same shape as pad 16, in some embodiments matching a shape and surface area; however, x-ray detectable layer 26 is slightly larger than pad 16 in length and width as shown. In some embodiments, x-ray detectable layer 26 may be up to, but not exceeding 100% of the surface area of pad 16. In some embodiments, x-ray detectable layer 26 may be greater than the surface area of pad 16.

FIG. 3B depicts an exemplary configuration comprising a much smaller x-ray detectable layer 26. In some embodiments, an amount of x-ray detectable material may be present in a much smaller volume of x-ray detectable layer 26. Therefore, as long as the flexibility is maintained, a smaller and/or thinner x-ray detectable layer 26 may be used. For example, x-ray detectable layer 26 may be smaller in size and shape than pad 16 and, in some embodiments, may include a thickness of between 0.25 mm and 0.40 mm or even thinner while maintaining a quantity of x-ray detectable material that is 1.0, 1.1, 1.2, 1.3, or more times greater than the detectable quantity of x-ray detectable material.

FIG. 3C depicts an exemplary configuration comprising a longer, yet narrower, x-ray detectable layer 26. The longer narrower configuration may provide greater lengthwise flexibility and may be beneficial to adhering to the user's skin during movement. The various configurations shown in FIGS. 3A-3C are exemplary illustrating that any size and shape of x-ray detectable layer 26 and metal detectable layer 28 may be used and x-ray detectable layer 26 and metal detectable layer 28 may be switched.

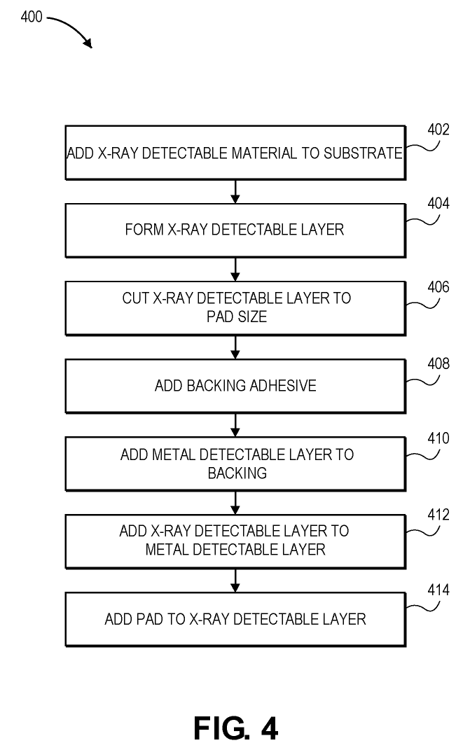

FIG. 4 depicts a flow chart illustrating an exemplary manufacturing and assembly process 400 of detectable bandage 10. At step 402, the x-ray detectable material may be added to the matrix as described in embodiments above. In some embodiments, the matrix may be EVA and the x-ray detectable material may be barium sulfate. The x-ray detectable material may be added in quantities based on the above- described optimization of flexibility, size and shape (e.g., thickness), detectability, and the like of x-ray detectable layer 26 and pad 16. The x-ray detectable material may be added and heated and mixed with the matrix until substantially isotropic. The isotropic layer provides a near even distribution of x-ray detectable material throughout the mixture of x- ray detectable layer 26.

At step 404, x-ray detectable layer 26 may be formed and cut to the size and shape determined in the optimization described above. In some embodiments, after the x-ray detectable material has been added to the inert matrix, the mixture may be heated to malleable temperature. The mixture may then by formed by extrusion, molding, pressing, cutting, and the like to a desired thickness and/or shape. In some embodiments, the desired thickness may be approximately 0.33 mm when the x-ray detectable material is barium sulfate, and the matrix is EVA. However, any thickness and dimensions described above may be imagined.

At step 406, the formed x-ray detectable layer 26 including the matrix and x-ray detectable material mixture may be processed. When the desired thickness (e.g., 0.33 mm) is achieved and cooled, the product may be cut and finished to the final size and shape of x-ray detectable layer 26. The final size and shape, in some embodiments, may be approximately the same as pad 16 or any of the above-described configurations. In some embodiments, the size and shape may match pad 16, or the size and shape may be larger or smaller as illustrated in FIGS. 3A-3D.

At step 408, backing adhesive layer 14 may be added to backing 12. Backing adhesive 18 may provide the attachment mechanism to attach detectable bandage 10 to the skin of the user. Backing adhesive 18 may comprise methacrylate, epoxy diacrylates, and the like as described above.

At step 410, metal detectable layer 28 may be disposed on backing 12 and attached, in some embodiments, with an adhesive. Metal detectable layer 28 may comprise any material that may be detectable by any metal detecting technique. In some embodiments, metal detectable layer 28 may comprise a thin flexible layer of tin, aluminum, copper, and the like (e.g., tin foil). In some embodiments, metal detectable layer 28 may be cut to a size and shape of pad 16 and/or a size and shape of x-ray detectable layer 26. The size, shape, and thickness, of metal detectable layer 28 may provide a high likelihood of detectability by standard metal detecting techniques in the industry.

At step 412, x-ray detectable layer 26 may be disposed on metal detectable layer 28 and attached with the adhesive. In some embodiments, x-ray detectable layer 26 may comprise a mixture of EVA and barium sulfate. The barium sulfate may be added in a quantity as a ratio with the EVA that provides a high likelihood of being detected by standard x-ray detection methods given the size and shape of x-ray detectable layer 26. In some embodiments, the thickness of x-ray detectable layer 26 may be based at least in part on a desired flexibility. Flexibility of x-ray detectable layer 26 may provide for longer and better attachment to the user's skin by allowing detectable bandage 10 to conform to shapes of the user's skin and structure and movements of the user. As such, detectable bandage 10 may stay attached longer than other bandages comprising x-ray detectable and metal detectable materials. For example, x-ray detectable layer 26 may be approximately 0.25-0.4 mm thick and approximately the size and shape of pad 16. X-ray detectable material may be added at a specific ratio to EVA to produce a high likelihood of detectability.

In some embodiments, x-ray detectable layer 26 may be disposed between metal detectable layer 28 and pad 16 as described herein. Alternatively, in some embodiments, metal detectable layer 28 may be disposed between x-ray detectable layer 26 and pad 16.

At step 414, pad 16 may be disposed on x-ray detectable layer 26 and attached by the adhesive. Pad 16 may be an absorbent material such as, for example, woven or nonwoven fabric, a polymer, rubber composite, and any combination thereof. Pad 16 may further be provided with any medicine (e.g., an antiseptic to aid in healing).

Although the present disclosure has been described with reference to the embodiments illustrated in the attached drawing figures, it is noted that equivalents may be employed, and substitutions made herein without departing from the scope of the present disclosure as recited in the claims.

Having thus described various embodiments of the present disclosure, what is claimed as new and desired to be protected by Letters Patent includes the following:

1. A detectable bandage, comprising:
an elastic backing layer;
a pad configured to be applied to a wound of a user contacting the user on a front side and comprising a back side opposite the front side;
a metal detectable layer comprising a metal detectable material disposed between the elastic backing layer and the pad;
an x-ray detectable layer comprising an x-ray detectable material, the x-ray detectable layer disposed between the elastic backing layer and the pad adjacent the metal detectable layer;
wherein the x-ray detectable layer covers approximately 100% of an area of the back side of the pad; and
an adhesive on the elastic backing layer configured to contact and adhere to a skin of the user to hold the detectable bandage on the skin when in use.

2. The detectable bandage of claim 1, wherein the metal detectable layer is disposed adjacent the elastic backing layer and the x-ray detectable material is disposed between the metal detectable layer and the pad.

3. The detectable bandage of claim 1, wherein the x-ray detectable material is barium sulfate.

4. The detectable bandage of claim 3,
wherein the x-ray detectable layer further comprises a matrix,
wherein the matrix comprises ethylene-vinyl acetate.

5. The detectable bandage of claim 4, wherein a thickness of the x-ray detectable layer is approximately 0.25 to 0.40 millimeters.

6. The detectable bandage of claim 1,
wherein the metal detectable material is a first metal detectable material; and
further comprising a second metal detectable material disposed in the elastic backing layer.

7. The detectable bandage of claim 1, wherein the metal detectable material comprises tin.

8. The detectable bandage of claim 1, wherein the x-ray detectable material covers at least a portion of the elastic backing layer adjacent the pad.

9. A detectable bandage, comprising:
an elastic backing layer;
a pad comprising a front side configured to be applied to a wound of a user and a back side opposite the front side;
a metal detectable layer comprising a metal detectable material disposed between the elastic backing layer and the pad;
an x-ray detectable layer comprising an x-ray detectable material, the x-ray detectable layer disposed between the elastic backing layer and the pad adjacent the metal detectable layer,
wherein the x-ray detectable layer comprises barium sulfate;
wherein the x-ray detectable layer covers less than 100% of an area of the back side of the pad; and
an adhesive on the elastic backing layer configured to contact and adhere to a skin of the user to hold the detectable bandage on the skin.

10. The detectable bandage of claim 9, wherein the metal detectable layer is disposed adjacent the elastic backing layer and the x-ray detectable material is disposed between the metal detectable layer and the pad.

11. The detectable bandage of claim 10,
wherein the x-ray detectable layer further comprises a matrix, and
wherein the matrix comprises ethylene-vinyl acetate.

12. The detectable bandage of claim 11, wherein the x-ray detectable material covers approximately an entire surface area of a back side of the pad.

13. The detectable bandage of claim 9, wherein a thickness of the x-ray detectable layer is approximately 0.25 to 0.40 millimeters.

14. The detectable bandage of claim 9, wherein the metal detectable material comprises tin.

15. A detectable bandage, comprising:
an elastic backing layer;
a pad configured to be applied to a wound of a user;
a metal detectable layer comprising a metal detectable material disposed between the elastic backing layer and the pad;
an x-ray detectable layer comprising an x-ray detectable material and a matrix comprising ethylene-vinyl acetate (EVA), the x-ray detectable layer disposed between the elastic backing layer and the pad adjacent the metal detectable layer,
wherein the x-ray detectable layer comprises barium sulfate; and
an adhesive on the elastic backing layer configured to contact and adhere to a skin of the user to hold the detectable bandage on the skin.

16. The detectable bandage of claim 15, wherein the metal detectable layer is disposed adjacent the elastic backing layer and the x-ray detectable material is disposed between the metal detectable layer and the pad.

17. The detectable bandage of claim 16, wherein the x-ray detectable material covers approximately 100% of a surface area of a back side of the pad.

18. The detectable bandage of claim 17, wherein a thickness of the x-ray detectable layer is in a range of 0.25 to 0.40 millimeters.

19. The detectable bandage of claim 18, wherein the metal detectable material comprises tin foil.

20. The detectable bandage of claim 19, wherein the x-ray detectable layer is an isotropic mixture of the EVA and the barium sulfate.
